(19) United States
(12) Patent Application Publication
(10) Pub. No.: US 2026/0325853 A1
(43) Pub. Date: Oct. 22, 2026

(54) IMPLANTABLE LEAD TORQUE TRANSFER TOOL

(71) Applicant: Pacesetter, Inc., Sylmar, CA (US)

(72) Inventors: Claire Franz, Los Angeles, CA (US); Wesley Alleman, Santa Clarita, CA (US); Cody Ledbetter, Santa Clarita, CA (US); Peter Fong, Valencia, CA (US); Keith Victorine, Valencia, CA (US)

(21) Appl. No.: 19/648,869

(22) Filed: Apr. 15, 2026

Related U.S. Application Data
(60) Provisional application No. 63/791,320, filed on Apr. 18, 2025.

(51) Int. Cl. A61N 1/372; A61N 1/05

(52) U.S. Cl. CPC A61N 1/372; A61N 1/057; A61N 2001/058

(57) ABSTRACT

A torque transfer tool includes a drive stylet and a lead-stylet locking tool. The drive stylet includes a stylet body extending along a longitudinal axis to a stylet tip, and a projection extending laterally outward from the stylet body. The lead-stylet locking tool includes a tool body having a body wall extending around a central lumen. The central lumen extends along the longitudinal axis from a distal tool end to a proximal tool end. The central lumen includes a connector body subsegment sized to receive a connector body of an implantable lead. The central lumen includes a pin subsegment sized to receive a connector pin of the implantable lead. A lateral space extends through the body wall from the central lumen to receive the projection of the drive stylet.

This application claims priority to U.S. Patent Application No. 63/791,320, entitled “IMPLANTABLE LEAD TORQUE TRANSFER TOOL” and filed on Apr. 18, 2025, which is hereby incorporated by reference in its entirety.

### BACKGROUND

### Field

The present disclosure relates to leads for conducting electrical signals to and from target tissue. More particularly, embodiments related to accessories used with implantable leads for delivering electrical charges to cardiac tissue are disclosed.

### Background Information

Cardiac pacing by an artificial pacemaker provides an electrical stimulation of the heart when its own natural pacemaker and/or conduction system fails to provide synchronized atrial and ventricular contractions at rates and intervals sufficient for a patient's health. Such antibradycardial pacing provides relief from symptoms and even life support for hundreds of thousands of patients. Cardiac pacing may also provide electrical overdrive stimulation to suppress or convert tachyarrhythmias, again supplying relief from symptoms and preventing or terminating arrhythmias that could lead to sudden cardiac death.

Conduction system pacing (CSP) can use implantable leads, such as pacing or defibrillation leads, to provide an electrical connection between the artificial pacemaker and a target location of the cardiac conduction system that is to be electrically stimulated. The target anatomy may be, for example, a His bundle of a heart. Cardiac pacing of the His bundle is clinically effective and advantageous by providing a narrow QRS affecting synchronous contraction of the ventricles. His bundle pacing in or near a membranous septum of a heart, however, has some drawbacks. Successful His bundle pacing cannot always be achieved. Pacing thresholds are often high, sensing is challenging, and success rates can be low.

CSP at the left bundle branch (LBB) is an alternative to His bundle pacing. Pacing at the LBB involves pacing past the His bundle toward the right ventricle apex. More particularly, a pacing site for LBB pacing is typically below the His bundle, on the interventricular septal wall. To achieve optimal results, the pacing site for physiological pacing at the LBB can be relatively deep in the interventricular septal wall, compared to the His bundle. For LBB area implantation, a physician implants a pacing lead by turning a lead body by hand to engage a distal end of the pacing lead deep into the septal wall. Torque is delivered down a length of the flexible lead body to drive the distal end forward.

### SUMMARY

Existing approaches for conduction system pacing (CSP) use existing implantable leads, such as pacing or defibrillation leads, which include a fixed or extendable helix to engage the target tissue. The target pacing site can be deep in the interventricular septal wall. For example, the target pacing site of left bundle branch (LBB) area pacing may be several centimeters deep in the interventricular septal wall. Therefore, a physician may be required to rotate and push an implantable lead to drive the implantable lead through the myocardial tissue to the target pacing site. Existing implantable leads are flexible by design, however, and the application of torque to a proximal end of the implantable lead can be absorbed in the flexible lead body. More particularly, the torsion can cause deformation, e.g., wrinkling, and torque build up over a lead length, and can reduce the torque that reaches the distal end of the pacing lead. Such deformation can be exacerbated by variations in tissue consistency, which can complicate the procedure for physicians. More particularly, the deformation and torque build up can stress lead components and interfere with lead placement. Thus, there is a need for an implantable lead or implantable lead system that efficiently transfers torque input at the proximal end by the physician to burrow a distal end of the implantable lead through the interventricular septal wall to the target tissue. Efficient torque transfer could make implantation more reliable and repeatable, regardless of the tissue consistency being encountered by the implantable lead, resulting in a more consistent implant behavior for the physician while also reducing a number of turns of the lead body required to achieve implantation.

A drive stylet is described. In an embodiment, the drive stylet includes a stylet body extending along a longitudinal axis to a stylet tip. The drive stylet includes a projection extending laterally outward from the stylet body.

A lead-stylet locking tool is described. In an embodiment, the lead-stylet locking tool includes a tool body including a body wall extending around a central lumen. The central lumen extends along a longitudinal axis from a distal tool end to a proximal tool end. The central lumen includes a connector body subsegment sized to receive a connector body of an implantable lead and a pin subsegment sized to receive a connector pin of the implantable lead. A lateral space extends through the body wall from the central lumen to receive a projection of a drive stylet.

A torque transfer tool is described. The torque transfer tool includes the drive stylet and the lead-stylet locking tool.

The above summary does not include an exhaustive list of all aspects of the present invention. It is contemplated that the invention includes all systems and methods that can be practiced from all suitable combinations of the various aspects summarized above, as well as those disclosed in the Detailed Description below and particularly pointed out in the claims filed with the application. Such combinations have particular advantages not specifically recited in the above summary.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings.

FIG. 1 is a diagrammatic cross-sectional view of a patient heart illustrating an example implantation of an implantable lead in a target anatomy, in accordance with an embodiment.

FIG. 2 is a perspective view of an implantable lead, in accordance with an embodiment.

FIG. 3 is a perspective view of a drive stylet inserting into an implantable lead, in accordance with an embodiment.

FIG. 4 is a sectional view of a drive mechanism of an implantable lead system, in accordance with an embodiment.

FIG. 5 is a perspective view of a torque transfer tool being loaded onto an implantable lead, in accordance with an embodiment.

FIG. 6 is a perspective view of a torque transfer tool rotating an implantable lead, in accordance with an embodiment.

FIG. 7 is a perspective view of a drive stylet, in accordance with an embodiment.

FIG. 8 is a perspective view of a torque transfer tool, in accordance with an embodiment.

FIG. 9 is a sectional view of a torque transfer tool, in accordance with an embodiment.

FIG. 10 is a sectional view of a torque transfer tool loaded on an implantable lead, in accordance with an embodiment.

FIG. 11 is a sectional view of a lead-stylet locking tool, in accordance with an embodiment.

FIG. 12 is a sectional view of a lead-stylet locking tool, in accordance with an embodiment.

FIG. 13 is a perspective view of a lead-stylet locking tool, in accordance with an embodiment.

FIG. 14 is a detail view of a retaining slot of a lead-stylet locking tool, in accordance with an embodiment.

FIG. 15 is a pictorial view of a torque transfer tool loaded on an implantable lead, in accordance with an embodiment.

FIG. 16 is a perspective view of a lead-stylet locking tool, in accordance with an embodiment.

FIG. 17 is an end view of a lead-stylet locking tool, in accordance with an embodiment.

FIG. 18 is a pictorial view of a torque transfer tool, in accordance with an embodiment.

FIG. 19 is a pictorial view of a torque transfer tool, in accordance with an embodiment.

FIG. 20 is a perspective view of a torque transfer tool, in accordance with an embodiment.

FIG. 21 is a perspective view of a torque transfer tool, in accordance with an embodiment.

FIG. 22 is a perspective view of a torque transfer tool, in accordance with an embodiment.

FIG. 23 is a perspective view of a torque transfer tool loaded on an implantable lead, in accordance with an embodiment.

FIG. 24 is a perspective, cutaway view of a torque transfer tool loaded on an implantable lead, in accordance with an embodiment.

### DETAILED DESCRIPTION

Embodiments describe a drive stylet, a lead-stylet locking tool, and a torque transfer tool comprising the drive stylet and the lead-stylet locking tool. The torque transfer tool may be used during implantation of cardiac pacing leads. For example, the torque transfer tool can assist in delivering cardiac pacing leads to a Left Bundle Branch (LBB). The torque transfer tool may, however, be used during implantation of pacing leads at other physiological sites, such as within the brain or spine, to name a few alternative applications.

In various embodiments, description is made with reference to the figures. However, certain embodiments may be practiced without one or more of these specific details, or in combination with other known methods and configurations. In the following description, numerous specific details are set forth, such as specific configurations, dimensions, and processes, in order to provide a thorough understanding of the embodiments. In other instances, well-known processes and manufacturing techniques have not been described in particular detail in order to not unnecessarily obscure the description. Reference throughout this specification to “one embodiment,” “an embodiment,” or the like, means that a particular feature, structure, configuration, or characteristic described is included in at least one embodiment. Thus, the appearance of the phrase “one embodiment,” “an embodiment,” or the like, in various places throughout this specification are not necessarily referring to the same embodiment. Furthermore, the particular features, structures, configurations, or characteristics may be combined in any suitable manner in one or more embodiments.

The use of relative terms throughout the description may denote a relative position or direction. For example, “distal” may indicate a first direction along a longitudinal axis of an implantable lead, a drive stylet, or an implantable lead system. Similarly, “proximal” may indicate a second direction opposite to the first direction. Such terms are provided to establish relative frames of reference, however, and are not intended to limit the use or orientation of the implantable lead, the drive stylet, or the implantable lead system to a specific configuration described in the various embodiments below.

In an aspect, a torque transfer tool can lock a drive stylet to an implantable lead, and may optionally temporarily convert the implantable lead from an extendable cardiac pacing lead into a fixed cardiac pacing lead. The locked stylet and lead system can therefore behave as a singular body in which torque transferred through the drive stylet is also transmitted through the implantable lead. For example, the torque can be transmitted from a stylet tip of the drive stylet to a distal end of the implantable lead. The drive stylet provides a rigid, e.g., stainless steel, member at a center of twist that improves torque transfer. More particularly, the torque transfer tool locks the drive stylet to the lead body, enabling the components to rotate together as a rigid member. Accordingly, the torque transfer tool makes implantation of the implantable lead easier and more reliable for physicians, for example, during conduction system pacing implantation in a left bundle branch area.

Several embodiments of a torque transfer tool are described below. The embodiments may be generally grouped into systems having different component parts. For example, some embodiments relate to a one-piece system, having a drive stylet with a permanently attached, e.g., integrated, lead-stylet locking tool. Some embodiments relate to a two-piece system, having a drive stylet with a separable lead-stylet locking tool. Other embodiments relate to a three-piece system, having a drive stylet with a multi-component, separable lead-stylet locking tool. Each of the systems described herein provides similar advantages. For example, the torque transfer tool can enable easier implantation deep into a septal wall for conduction system pacing by providing improved torque transfer, which requires fewer turns of a lead body to result in greater torque output. The improved torque transfer can reduce a risk of damage to the pacing lead. Additional advantages include the accommodation of an electrical connection to the drive stylet for unipolar pacing through a pacing system analyzer (PSA) clip. Furthermore, the embodiments described below can be lightweight and have a minimal form factor to avoid weighing down the implantable lead during the implantation procedure.

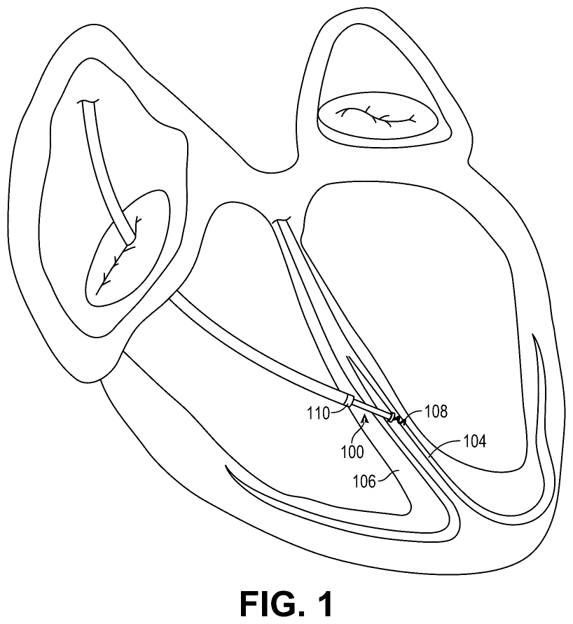

Referring to FIG. 1, a diagrammatic cross-sectional view of a patient heart illustrating an example implantation of an implantable lead in a target anatomy is shown in accordance with an embodiment. An implantable lead 100, e.g., a pacing lead or an implantable cardioverter defibrillator lead, may be a transvenous lead that can be delivered to a target anatomy to conductively pace the target anatomy. The target anatomy can include a septal wall 106 of a heart and/or a LBB 104 in the septal wall 106.

The implantable lead 100 may be any type of pacing lead. For example, the implantable lead 100 can include an extendable or retractable helix, or a fixed helix. Furthermore, the implantable lead 100 may be unipolar or bipolar. The implantable lead 100 may be of a high voltage or a low voltage lead type. Accordingly, the illustrations and descriptions with respect to lead structure are provided herein by way of example and not limitation.

In an embodiment, the implantable lead 100 can include an active fixation element, e.g., a helical fixation element 108. The helical fixation element 108 can include an electrode, which may be fixed or extendable, relative to a ring electrode 110. The implantable lead 100 can be driven into the interventricular septum to engage and anchor the implantable lead 100 to the tissue. The helical fixation element 108 can be an active electrode, and thus, can pace the tissue. When the helical fixation element 108 is located at the LBB 104, the ring electrode 110 can be located at or on a surface of the septal wall 106. The ring electrode 110 can be a passive or an active electrode. Accordingly, the electrodes can facilitate optimal placement of each electrode to provide a more robust multisite pacing modality.

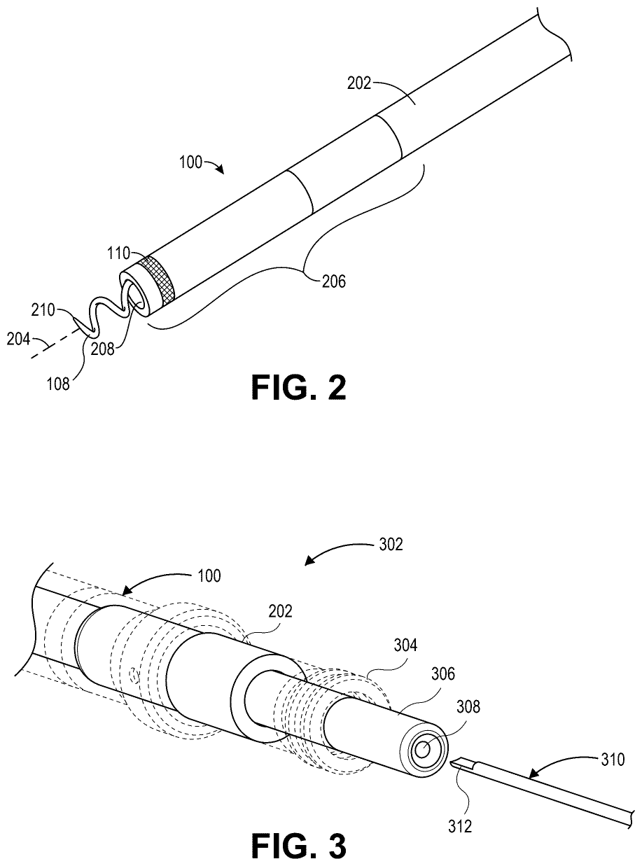

Referring to FIG. 2, a perspective view of an implantable lead is shown in accordance with an embodiment. The implantable lead 100 can include a lead body 202 extending along a central axis 204 from a proximal lead portion (FIG. 3) to a distal lead portion 206. The lead body 202 can include a central lumen 208 in the distal lead portion 206. The central lumen 208 can house a portion of the helical fixation element 108. More particularly, the helical fixation element 108 can extend distally from the central lumen 208. In an embodiment, the helical fixation element 108 is fixed, with a distal tip permanently extended beyond an end of the lead body 202. In an embodiment, the helical fixation element 108 is extendable, with the distal tip capable of extending or retracting from the central lumen 208, or relative to a distal end of the lead body 202.

As described above, the implantable lead 100 can have one or more electrodes, such as the helical fixation element 108 and/or the ring electrode 110. The electrode(s) can be driven into target tissue or pacing. For example, the distal tip of the helical fixation element 108 can be screwed into the interventricular septal wall 106 to contact and pace a LBB 104.

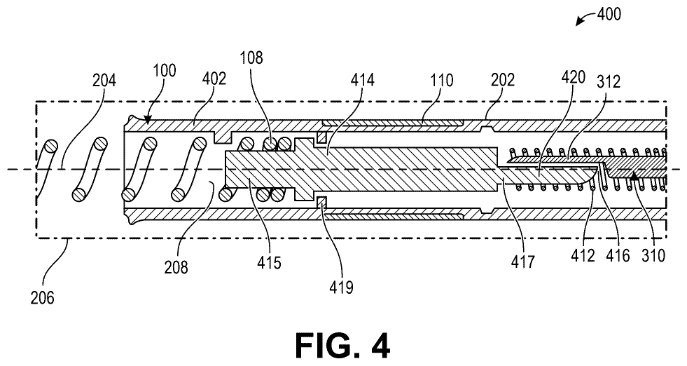

Referring to FIG. 3, a perspective view of a drive stylet 310 inserting into an implantable lead 100 is shown in accordance with an embodiment. A proximal lead portion 302 of the implantable lead 100 can include a seal 304 and a connector pin 306. The seal 304 can be at a proximal end of the lead body 202, and can create a seal between the implantable lead 100 and a header of a biostimulator when the connector pin 306 is plugged into an electrical port of the biostimulator. The connector pin 306 can conduct electrical signals between the electrical port and the electrode(s) of the implantable lead 100. For example, the connector pin 306 can be electrically connected to an inner coil (FIG. 4) to conduct electrical signals to the helical fixation element 108. In an embodiment, the connector pin 306 is rotatably mounted in the lead body 202 and can transmit torque to the inner coil to rotate a drive coupling (FIG. 4) for extending or retracting the helical fixation element 108 relative to the lead body 202.

In an embodiment, the connector pin 306 can include a pin lumen 308 extending along the central axis 204 into the lead body 202. More particularly, the pin lumen 308 can extend through the connector pin 306 into a coil lumen of the inner coil to provide a pathway to a drive coupling, as described below. For example, at an operation, a drive stylet 310 can be inserted into the pin lumen 308 and advanced through the implantable lead 100 to engage a drive shaft of the drive coupling.

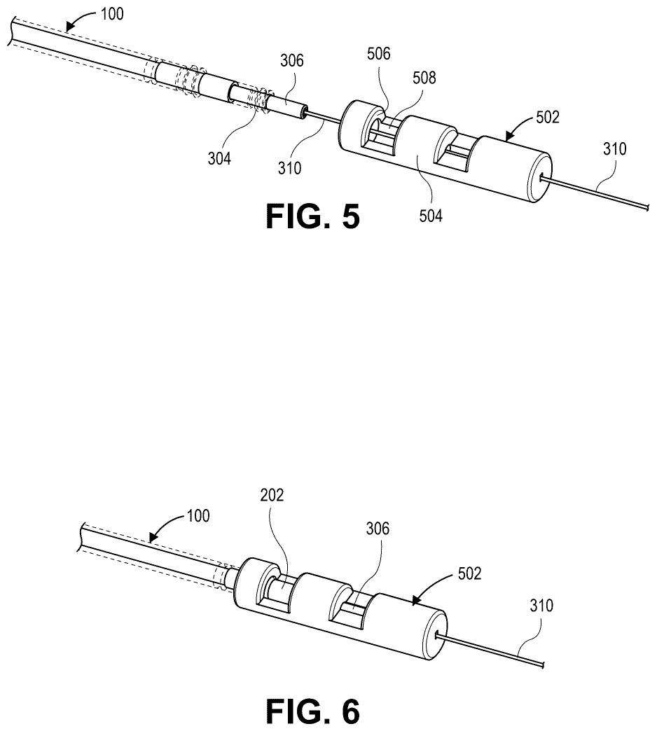

As described above, the drive stylet 310 can extend along a central axis 204 from a proximal shaft end (not shown) to the distal shaft end. The drive stylet 310 can be an elongated member, e.g., a cylindrical wire, sized to slide freely within the pin lumen 308 and the coil lumen of the implantable lead 100. The drive stylet 310 can be formed from a metal or another material having a high torsional stiffness. Furthermore, the drive stylet 310 can include a stylet tip 312, which may be shaped to engage the drive coupling of the implantable lead 100. Accordingly, an operator can twist the drive stylet 310 to transmit torque through the drive stylet 310 to the stylet tip 312 and the implantable lead 100. Furthermore, when the drive stylet 310 is locked to the implantable lead 100 via a lead-stylet locking tool (FIG. 5), as described below, the torque can transmit directly through the implantable lead 100 and the drive stylet 310 to the helical fixation element 108 to burrow into target tissue.

Referring to FIG. 4, a sectional view of a drive mechanism of an implantable lead system is shown in accordance with an embodiment. An implantable lead system 400 includes the implantable lead 100 and the drive stylet 310. The drive stylet 310 can engage the implantable lead 100 to transfer torque and drive the implantable lead 100 into the target tissue.

The lead body 202 can be further divided into individual components. In an embodiment, the lead body 202 includes an outer jacket 402 having a tubular, elongate structure. The outer jacket 402 may, for example, be an insulating jacket surrounding an outer coil (not shown). More particularly, the outer coil can include a coil extending from the proximal portion 302 of the implantable lead 100 to a distal lead portion 206 of the implantable lead 100 (shown in cross-section in FIG. 4), and the outer jacket 402 can be a silicone sleeve or layer mounted on the outer coil. The outer coil may be formed from an electrically conductive material to conduct sensing or stimulation, e.g., pacing, pulses from an external stimulation, e.g., pacing, device to the ring electrode 110. Accordingly, the outer jacket 402 can insulate the outer coil from a surrounding environment over a length that the outer jacket 402 covers the outer coil. In combination, the outer jacket 402 and the outer coil can form the lead body 202 having the central lumen 208 through which an inner lead subassembly extends.

The inner lead subassembly can include a tubular, elongate structure. For example, the inner lead subassembly can have an inner coil 412 extending helically about, and longitudinally along, the central axis 204. More particularly, the inner coil 412 can extend through the lead body 202 along the central axis 204, e.g., within the central lumen 208. The inner coil 412 may be formed from an electrically conductive material, and thus, can conduct electrical stimulation pulses from the external stimulation device to distal structures that the inner coil 412 electrically communicates with, e.g., the helical fixation element 108.

The implantable lead 100 can include a drive coupling 414. The drive coupling 414 can have a shaft structure, e.g., a rotationally symmetric structure, including lengths to engage surrounding structures. For example, the drive coupling 414 can include a distal boss 415, e.g., a mounting prong, to engage the helical fixation element 108. More particularly, the helical fixation element 108 can have a helix lumen extending through the helical coil (the helical coil can extend about the helix lumen) and the helical fixation element 108 can be mounted on the drive coupling 414 such that the distal boss 415 is disposed within the helix lumen.

The drive coupling 414 may also be coupled to the inner coil 412. For example, the inner coil 412 can have a coil lumen 416, and a proximal boss 417, e.g., a mounting segment, of the drive coupling 414 can extend into the coil lumen 416. More particularly, the drive coupling 414 can be disposed in the coil lumen 416 and connected to the inner coil 412 to secure the inner coil 412 to the drive coupling 414.

Continuing to refer to FIG. 4, via the physical connections described above, the inner coil 412 can be electrically connected to the drive coupling 414 and the helical fixation element 108 can be electrically connected to the drive coupling 414. The structures may be conductive and, thus, pacing and sensing pulses may be communicated between the helical fixation element 108 and the inner coil 412 via the drive coupling 414.

The drive coupling 414 may be fixed or rotatable relative to the lead body 202. In an embodiment, the drive coupling 414 is fixed. For example, the drive coupling 414 can have an outer surface that is interconnected with the outer jacket 402 of the implantable lead 100. Accordingly, when torque is applied to the outer jacket 402 or the inner coil 412, such torque can be transmitted to the drive coupling 414. As described below, torque may also be delivered directly to the drive coupling 414, e.g., by engagement between mating drive surfaces of the drive coupling 414 and the drive stylet 310. The interconnection between the drive coupling 414 and the lead body 202 may be via an adhesive or thermal bond. Alternatively, the components may be unitarily formed, e.g., the lead body 202 can be molded onto or heat shrunk around the drive coupling 414.

In an embodiment, the drive coupling 414 is rotatable relative to the lead body 202. The drive coupling 414 may be rotatably mounted in the central lumen 208. For example, the drive coupling 414 can have an outer surface that is seated within or against a collar 419 of the lead body 202. The outer surface can rotatably slide within or against the collar 419 about the central axis 204 when torque is applied to the drive coupling 414. Such torque can be applied to the drive coupling 414 by the inner coil 412 or the drive stylet 310. In an embodiment, the inner coil 412 is coupled to the drive coupling 414. For example, the drive coupling 414 may be disposed in a coil lumen 416 of the inner coil 412, and the inner coil 412 can be attached to the outer surface of the drive coupling 414. The attachment between the inner coil 412 and the drive coupling 414 can transmit torque from the inner coil 412 to the drive coupling 414. When the inner coil 412 is rotated within the central lumen 208 of the lead body 202, the rotation is transmitted to the drive coupling 414.

In an embodiment, torque can be transmitted to the drive coupling 414 from a proximal end of the implantable lead 100 via the drive stylet 310. The drive coupling 414 can include a drive shaft 420 to receive a torque input from the drive stylet 310. More particularly, the stylet tip 312 of the drive stylet 310 can engage a surface of the drive shaft 420. The drive stylet 310 can be rotated to torque the drive shaft 420 and rotate the drive coupling 414. Accordingly, the drive coupling 414 can torque the helical fixation element 108 and/or the lead body 202 to burrow the implantable lead 100 into the target tissue.

Referring to FIG. 5, a perspective view of a torque transfer tool being loaded onto an implantable lead 100 is shown in accordance with an embodiment. A physician can attach a lead-stylet locking tool 502 to the connector pin 306 on the proximal end of the implantable lead 100. The lead-stylet locking tool 502, also referred to herein as a “locking tool” herein, can be a component of the implantable lead system 400. More particularly, the lead-stylet locking tool 502 can lock the implantable lead 100, e.g., the connector pin 306, to the drive stylet 310. For example, the lead-stylet locking tool 502 can be packaged or used with the implantable lead 100 and the drive stylet 310. The lead-stylet locking tool 502 can function to lock the implantable lead 100 to the drive stylet 310. More particularly, as described below, the lead-stylet locking tool 502 can receive the lead body 202 and the drive stylet 310 to fix the lead body 202 relative to the drive stylet 310. The lead-stylet locking tool 502 may be integral with, or separate from, the drive stylet 310.

In an embodiment, the lead-stylet locking tool 502 includes a tool body 504. The tool body 504 can have a distal section configured to grip the implantable lead 100. For example, the distal section can have an internal bore sized to press fit onto the seal 304 or the connector pin 306. More particularly, the tool body 504 can have a body wall 506 extending around a tool lumen 508. The distal section can have a portion of the tool lumen 508 sized to form a friction fit with a portion of the implantable lead 100. A proximal section of the lead-stylet locking tool 502 can be mounted on the drive stylet 310. For example, the proximal section can have a portion of the tool lumen 508 sized to form a friction fit with, or adhere to, the drive stylet 310.

Referring to FIG. 6, a perspective view of a torque transfer tool rotating an implantable lead 100 is shown in accordance with an embodiment. Via the stylet tip 312 to drive coupling 414 engagement described above, the drive stylet 310 can transmit torque directly to the drive shaft 420 and, thus, the helical fixation element 108. It may also be advantageous to lock the drive stylet 310 to the lead body 202 such that the implantable lead 100 and the drive stylet 310 operate as a unitary body. For example, when the helical fixation element 108 is not fixed to the lead body 202, e.g., is extendable, rotating the helical fixation element 108 by the drive stylet 310 may not rotate the lead body 202 and can limit device deliverability. Similarly, even when the helical fixation element 108 is fixed to the lead body 202, delivering torque directly to the drive shaft 420 without also rotating the proximal lead portion 302 may cause wrinkling or torque buildup in the lead body 202, which can adversely affect deliverability. Accordingly, the lead-stylet locking tool 502 can constrain or lock the drive stylet 310 relative to the lead body 202. More particularly, when the lead-stylet locking tool 502 is mounted on the implantable lead 100 and the drive stylet 310, the lead-stylet locking tool 502 receives the lead body 202 and the drive stylet 310 to fix the lead body 202 relative to the drive stylet 310 within the tool lumen 508. The lead-stylet locking tool 502 can constrain the drive stylet 310 relative to the lead body 202, both laterally and longitudinally relative to the central axis 204, and rotationally about the central axis 204. In other words, the lead-stylet locking tool 502 can fix the drive stylet 310 relative to the lead body 202. Accordingly, the embodiments described below lock the drive stylet 310 to the implantable lead 100 to allow torque to be directly applied through a center of the assembly during lead implantation.

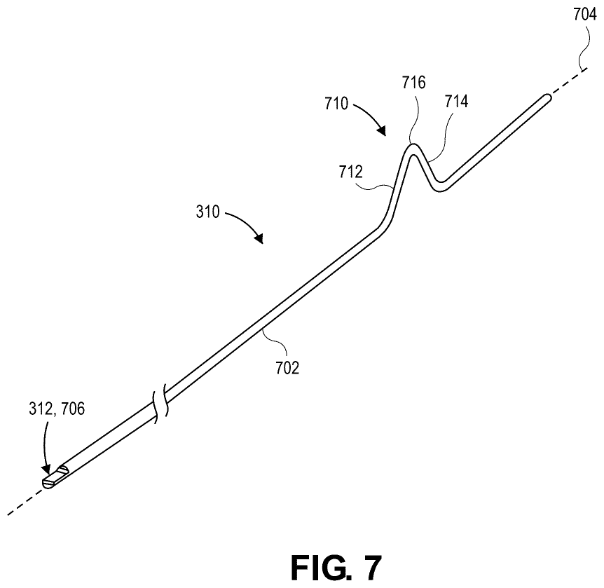

Referring to FIG. 7, a perspective view of a drive stylet 310 is shown in accordance with an embodiment. The drive stylet 310 can include an elongated body. More particularly, in an embodiment, the drive stylet 310 includes a stylet body 702 extending along a longitudinal axis 704 to the stylet tip 312. The longitudinal axis 704 can be aligned with the central axis 204 of the implantable lead 100 when the drive stylet 310 is inserted into the pin lumen 308.

The drive stylet 310 can have a distal stylet portion, designed to be inserted into the implantable lead 100 and to engage with the drive coupling 414, and a proximal stylet portion, designed to connect to the lead-stylet locking tool 502. In an embodiment, the distal stylet portion has the stylet tip 312. The stylet tip 312 may include a driver head 706 to facilitate engagement to the drive coupling 414. The driver head 706 may, for example, include a flat head, Philips-type, or other symmetrical driver pattern. Alternatively, the driver head 706 may be asymmetrical, e.g., have an offset semi-circular pattern as shown in FIG. 7. In any case, the driver head 706 can match a corresponding socket in the drive coupling 414 to allow the driver head 706 to securely engage the drive coupling 414. Torque may therefore be efficiently and directly transmitted through the stylet body 702 to the driver head 706 and drive coupling 414 to cause rotation of the helical fixation element 108.

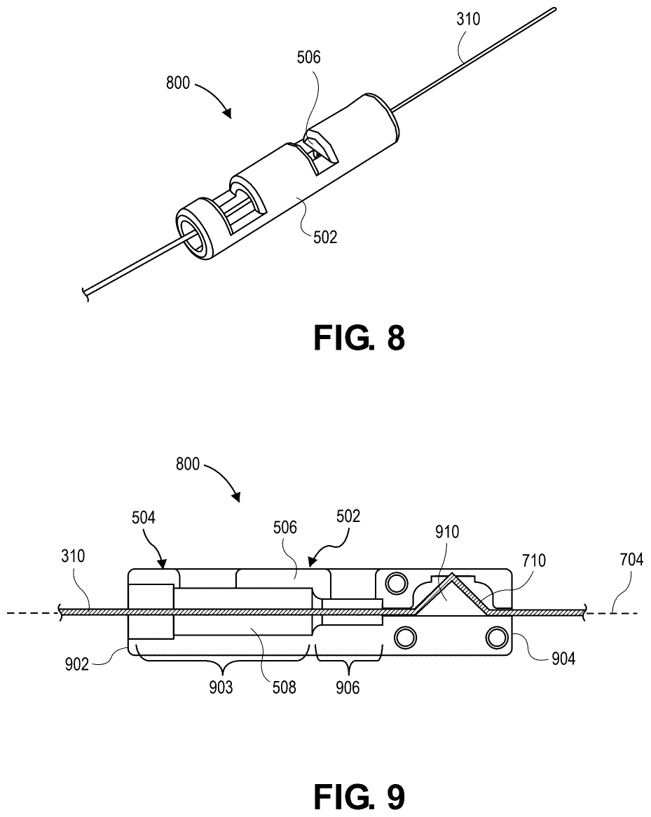

The proximal stylet portion can include a portion of the drive stylet 310 that engages the lead-stylet locking tool 502 to form a torque transfer tool (FIG. 8), as described below. The proximal stylet portion can be a portion of the drive stylet 310 that remains outside of the lead body 202 when the stylet tip 312 is engaged with the drive coupling 414. Accordingly, the proximal stylet portion may be substantially shorter than the distal stylet portion, which resides within the coil lumen 416 during such engagement. For example, the distal stylet portion may be at least ten times longer than the proximal stylet portion.

In an embodiment, the proximal stylet portion includes a projection 710 extending laterally outward from the stylet body 702. The projection 710 can include a jog in the stylet body 702, a separate component, such as a collar or a weld spot, or any other feature that protrudes radially outward from the longitudinal axis 704. Alternatively, rather than the projection 710, the proximal stylet portion can include an indentation, e.g., a circumferential recess, which extends radially inward toward the longitudinal axis 704 relative to an outer surface of the stylet body 702. In any case, the projection 710 (or indentation) provides a surface feature that can enlarge a transverse dimension of the stylet and engage the lead-stylet locking tool 502 to restrict relative motion between the lead-stylet locking tool 502 and the drive stylet 310 in one or more directions, e.g., longitudinally, laterally, or rotationally.

In an embodiment, the projection 710 includes a bend in the stylet body 702. The bend can jut outward from the longitudinal axis 704 and return inward toward the longitudinal axis 704. For example, the projection 710 can include a distal projection portion 712 joined to a proximal projection portion 714 port radially outward from the longitudinal axis 704. The distal projection portion 712 can be a portion of the wire form that juts outward and the proximal projection portion 714 can be a portion of the wire form that juts inward (or vice versa depending on perspective). The projection portions 712, 714 can be joined at a projection apex 716, which may be a radially outward most location, relative to the longitudinal axis 704, of the projection 710.

The projection 710 may be longitudinally compressible to allow portions of the stylet body 702 on either side of the projection 710 to move relative to each other. For example, the projection 710 illustrated in FIG. 7 can have a V-shape formed by the distal projection portion 712 and the proximal projection portion 714 connected at the projection apex 716. The V-shape can have an angle between the projection portions 712,714 that increases or decreases when a tensile or compressive force is applied to the projection 710. Accordingly, the stylet body 702 portions can move relative to each other, and a length of the projection 710 can increase or decrease. As described below, decreasing the length of the projection 710 can allow the projection 710 to be slipped into a slot within the lead-stylet locking tool 502 to form a torque transfer tool. Such a tool and others are now described. In another embodiment, the projection 710 can have a U-shape formed by the distal projection portion 712, the proximal projection portion 714, and an intermediate bridging or interconnection portion (not shown).

Referring to FIG. 8, a perspective view of a torque transfer tool 800 is shown in accordance with an embodiment. The torque transfer tool 800 includes the drive stylet 310 extending through lead-stylet locking tool 502. More particularly, the lead-stylet locking tool 502 may be mounted on, either permanently or temporarily, the drive stylet 310. For example, the lead-stylet locking tool 502 can be integrated with the drive stylet 310 to form the one-piece system, or separable from the drive stylet 310 to form the two-piece system. Embodiments of each are described below. In any case, when the torque transfer tool 800 is formed by the components, the projection 710 of the drive stylet 310 can be contained, at least partly, within the body wall 506 of the lead-stylet locking tool 502.

Referring to FIG. 9, a sectional view of a torque transfer tool 800 is shown in accordance with an embodiment. The torque transfer tool 800 includes the lead-stylet locking tool 502 containing at least a portion of the drive stylet 310. More particularly, in an embodiment, the body wall 506 extends around a tool lumen 508 of the lead-stylet locking tool 502, and the drive stylet 310 extends through the tool lumen 508. The projection 710 of the drive stylet 310 may also be at least partly contained within the lead-stylet locking tool 502. For example, projection 710 can extend laterally outward from the stylet body 702 toward the body wall 506.

The tool lumen 508 can extend along the longitudinal axis 704 from a distal tool end 902 to a proximal tool end 904. The projection 710 of the drive stylet 310, when the drive stylet 310 is engaged with the lead-stylet locking tool 502, can be located longitudinally between the distal tool end 902 and the proximal tool end 904. For example, in an embodiment, the tool lumen 508 includes several subsegments intended to receive the implantable lead 100, and the projection 710 can be located proximal to the subsegments, e.g., longitudinally between the subsegments and the proximal tool end 904.

In an embodiment, the tool lumen 508 subsegments include a connector body subsegment 903 distal to a pin subsegment 906. The connector body subsegment 903 can be sized to receive a connector body (a proximal portion of the lead body 202) of the implantable lead 100. Similarly, the pin subsegment 906 can be sized to receive the connector pin 306 of the implantable lead 100. The tool lumen 508 subsegments may be sized to receive the lead components with various degrees of friction fit. More particularly, the connector body subsegment 903 can be sized to receive the connector body in a first friction fit, and the pin subsegment 906 can be sized to receive the connector pin 306 in a second friction fit that is the same or different than the first friction fit(s). For example, the second friction fit can be tighter than the first friction fit. Accordingly, when mounted on the implantable lead 100, the locking tool 502 can engage the lead components to provide friction fit that locks the tool body 504 to the lead body 202 and/or connector pin 306. Such frictional engagement is described in U.S. patent application Ser. No. 18/100,532, titled “Pacing Lead Conversion Tool” and filed on Jan. 23, 2023, which published as U.S. Patent Publication No. 2023/0347142, the contents of which are incorporated by reference herein.

In an embodiment, the lead-stylet locking tool 502 includes a lateral space 910 extending through the body wall 506 from the tool lumen 508. The lateral space 910 can receive the projection 710 of the drive stylet 310. More particularly, the projection 710 can extend radially outward from the tool lumen 508 into the lateral space 910. The lateral space 910 may include an open cavity or may be filled (either with the same material as the body wall 506 or an additional material), as described in the various embodiments below. In any case, the extension of the projection 710 into the lateral space 910 can cause the drive stylet 310 to interfere with one or more surfaces of the lead-stylet locking tool 502, thereby limiting movement of the drive stylet 310 relative to the lead-stylet locking tool 502 and allowing the lead-stylet locking tool 502 to constrain the drive stylet 310 relative to the lead body 202 or connector pin 306 when the lead components are engaged with the tool body 504.

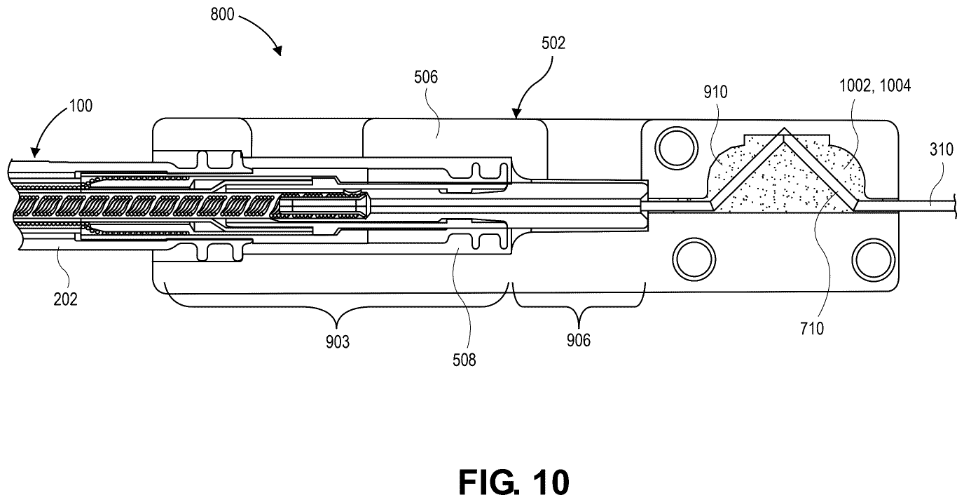

Referring to FIG. 10, a sectional view of a torque transfer tool 800 loaded on an implantable lead 100 is shown in accordance with an embodiment. In an embodiment, the lateral space 910 of the lead-stylet locking tool 502 includes a filler 1002. For example, the space may be entirely filled, e.g., with a same molding material used to form the remainder of the tool body 504. More particularly, the tool body 504 can be overmolded onto the drive stylet 310. In such case, the filler 1002, which may be a portion of the tool body 504 molding material, can be in conforming contact with the projection 710. As used herein, “conforming contact” refers to a relationship in which a first element surrounds a second element such that the first element is in immediate contact with an outer surface of the second element. For example, the filler 1002 can encapsulate and surround the projection 710, in immediate contact with an outer surface of the projection 710, such that the tool body 504 and the projection 710 are completely locked together. The encapsulation of the projection 710 by the filler 1002 can provide conforming contact between the components. The wire form can therefore fit within the filled lateral space 910 and be immovable in any direction relative to the tool body 504.

In an embodiment, rather than being overmolded onto the projection 710, the wire form can fit within the lateral space 910 and an encapsulant 1004 can surround the projection 710 within the tool body 504. As used herein, an “encapsulant” refers to a material that surrounds and encapsulates a component, thereby securing the component in place. The encapsulant 1004 may be an adhesive, an epoxy, a potting material, or another material, which is different from the tool body 504 material, and which is filled into the lateral space 910 to surround and encapsulate the projection 710. As in the overmolded embodiment, the encapsulant 1004 can be in conforming contact with the projection 710 to secure the projection 710 within the lateral space 910 to resist movement of the drive stylet 310 relative to the tool body 504.

It will be appreciated that, in both the overmolded and the encapsulant surrounded versions of the torque transfer tool 800, the torque transfer tool 800 can be a one-piece system having the lead-stylet locking tool 502 permanently fixed to the drive stylet 310. More particularly, the drive stylet 310 and the lead-stylet locking tool 502 can be integrated into a unitary assembly. The lead-stylet locking tool 502 may therefore be loaded onto the implantable lead 100 to lock the drive stylet 310 to the lead body 202. For example, the portion of the implantable lead 100 held within the connector body subsegment 903 and the pin subsegment 906 of the tool lumen 508 can be in a press fit with an inner surface of the tool body 504 and, accordingly, the lead-stylet locking tool 502 can resist relative motion between the implantable lead 100 and the drive stylet 310.

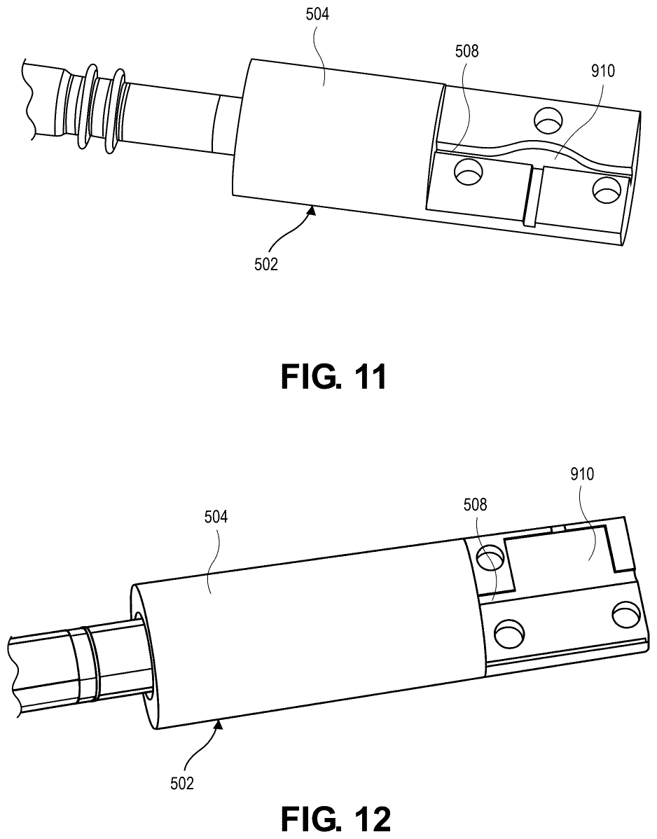

Referring to FIG. 11, a sectional view of a lead-stylet locking tool 502 is shown in accordance with an embodiment. The torque transfer tool 800 can have a two-piece system. More particularly, the lead-stylet locking tool 502 may be separable from the drive stylet 310. In an embodiment, the locking tool 502 can include a portion that can be assembled onto the drive stylet 310. The drive stylet 310 is not represented in FIG. 11, but it will be appreciated that the drive stylet 310 can be placed through the tool lumen 508 of the lead-stylet locking tool 502 to be loaded through the lateral space 910. The lateral space 910 can have a wave-like, or curved, form that includes a width that is greater than the tool lumen 508. Accordingly, the drive stylet 310 can be tracked through the tool lumen 508 until the projection 710 aligns with and expands radially outward within the lateral space 910. The lateral space 910 may optionally be filled with the encapsulant 1004 to secure the projection 710. Alternatively, the resistance between the projection 710 and the walls of the lateral space 910 can be sufficient to keep the projection 710 in place within the lateral space 910. In any case, a cap (not shown) may be placed over the tool body 504 that is shown in FIG. 11 to close the tool lumen 508 and lateral space 910, and to secure the drive stylet 310 relative to the tool body 504. Accordingly, the lead-stylet locking tool 502 can include a first tool portion (shown in FIG. 11) having a first cavity (the lateral space 910) and, optionally, a second tool portion, e.g., a cap, (not shown) having a second cavity (a space mirroring the lateral space 910). The tool portions can combine to form the lateral space 910 that retains the projection 710.

Referring to FIG. 12, a sectional view of a lead-stylet locking tool 502 is shown in accordance with an embodiment. An alternative lateral space 910 is shown, which can have a rectangular shape. The rectangular shape, like the wave-like shape shown in FIG. 11, can have a width that exceeds a diameter of the tool lumen 508. The drive stylet 310 can therefore be loaded into the tool lumen 508 to position the projection 710 in the lateral space 910. Movement of the drive stylet 310 relative to the tool body 504 can be resisted by the cavity surfaces (and, optionally, encapsulant 1004 loaded into the lateral space 910). A second tool portion (not shown) can then be loaded onto the first portion to complete the lead-stylet locking tool 502 that holds the drive stylet 310. The two-part locking tool 502 allows the physician to use the lead-stylet locking tool 502 with very minimal set up. The physician can simply slide the drive stylet 310 into the connector pin 306 and apply the lead-stylet locking tool 502 over the connector pin 306 to fix the drive stylet 310 to the proximal end of the lead 100.

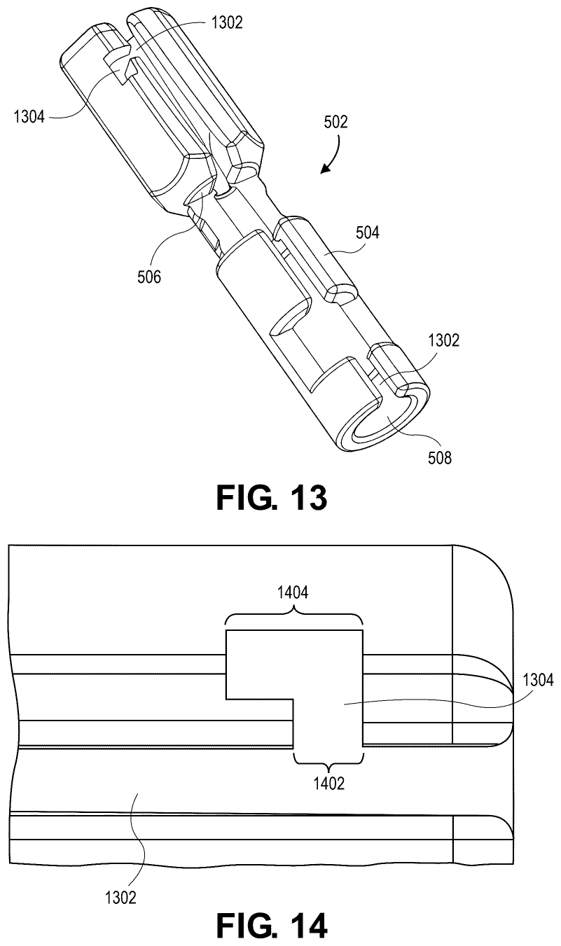

Referring to FIG. 13, a perspective view of a lead-stylet locking tool 502 is shown in accordance with an embodiment. Another two-piece system includes the lead-stylet locking tool 502 that can be temporarily coupled with the drive stylet 310. More particularly, the drive stylet 310 can be loaded into the lead-stylet locking tool 502 and then removed from the lead-stylet locking tool 502 at the convenience of the physician.

The drive stylet 310, such as the drive stylet 310 shown in FIG. 7, can be loaded into the lead-stylet locking tool 502 through a side slot 1302 in the lead-stylet locking tool 502. The side slot 1302 can extend through the body wall 506 of the lead-stylet locking tool 502. More particularly, the side slot 1302 can extend radially outward from the tool lumen 508 through the body wall 506 to a surrounding environment. The side slot 1302 can provide the lateral space 910 into which the projection 710 can extend when the lead-stylet locking tool 502 is loaded onto the drive stylet 310.

In an embodiment, the lateral space 910, which received the projection 710 of the drive stylet 310, may include a retaining slot 1304 to receive the projection 710. The retaining slot 1304 can be a feature, such as an L-shaped slot or T-shaped slot, into which the projection 710 can be inserted to lock the drive stylet 310 to the tool body 504. More particularly, the retaining slot 1304 can receive the projection 710 in a lateral direction and then hold the projection 710 in place to limit movement of the drive stylet 310 relative to the tool body 504.

The retaining slot 1304 can have a first segment that the projection 710 passes through, from the side slot 1302, to engage with a second segment that holds the projection 710. The first segment may include a circumferential slot segment (FIG. 14). The circumferential slot segment can extend laterally into the tool body 504 in a circumferential direction (relative to the longitudinal axis). By contrast, the second segment may include a longitudinal slot segment (FIG. 14). The longitudinal slot segment can extend longitudinally from an end of the circumferential slot segment, e.g., either distally or proximally.

In an embodiment, relative dimensions of the retaining slot 1304 segments may facilitate passage and/or capture of the projection 710 of the drive stylet 310. For example, the circumferential slot segment may be narrower than the longitudinal slot segment. More particularly, a width of the circumferential slot segment in the longitudinal direction can be less than a width of the longitudinal slot segment in the longitudinal direction. The narrower circumferential slot segment can receive the projection 710 in a compressed state, as described below, and the wider longitudinal slot segment can receive and retain the projection 710 in an expanded state.

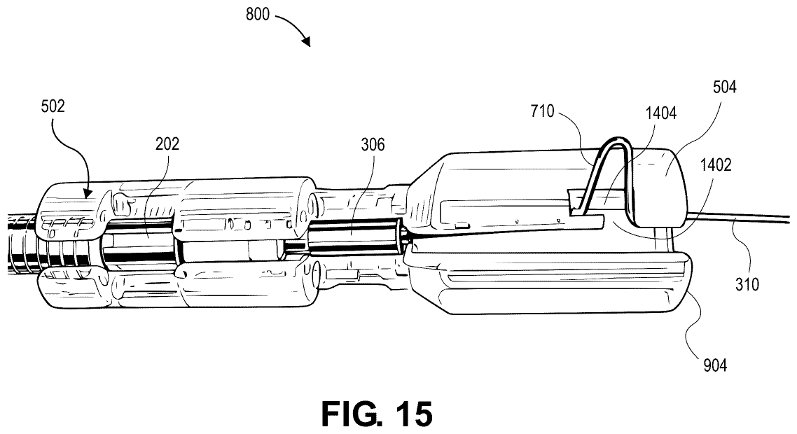

Referring to FIG. 14, a detail view of a retaining slot of a lead-stylet locking tool 502 is shown in accordance with an embodiment. In FIG. 15 below, the wire form is shown in the expanded state, retained within the longitudinal slot segment 1404, after passing laterally through the circumferential slot segment 1402. Referring back to FIG. 14, to facilitate such insertion, the spring wire form can be compressed to slide into the retaining slot 1304. The compressive force on the spring wire can reduce the length of the projection 710, e.g., to less than the width of the circumferential slot segment 1402, to allow the projection 710 to slide through such segment. The V-shape of the projection 710 can then be rotated through the circumferential slot segment 1402 into the longitudinal slot segment 1404. When the projection 710 is aligned with the longitudinal slot segment 1404, the compressive force can be removed, allowing the spring wire form to expand. More particularly, the length of the projection 710 can increase, allowing the V-shape of the projection 710 to widen and allowing the distal projection portion 712 to slide forward into the longitudinal slot segment 1404. Accordingly, the projection 710 is secured in place and translation of the drive stylet 310 relative to the lead body 202 can be prevented. More particularly, the retaining slot 1304 can lock the drive stylet 310 in a longitudinal and rotational direction relative to the tool body 504. The locked bodies, the drive stylet 310 and the lead-stylet locking tool 502, can form the torque transfer tool 800.

The use of a V-shaped projection 710 can accommodate a variety of tolerance interactions between the implantable lead 100 and the locking tool 502. For example, separate drive stylets 310 and/or lead-stylet locking tools 502 can be selected for use, depending on the implantable lead 100 being used, providing for a system that is suitable to many procedures. As a result, overall system costs can be reduced by ensuring functionality without requiring additional assembly tolerances on the implantable lead 100.

Referring to FIG. 15, a pictorial view of a torque transfer tool 800 loaded on an implantable lead 100 is shown in accordance with an embodiment. Stylets used for lead implantation commonly have a knob at a proximal end of the drive stylet 310 to allow a physician to manipulate, e.g., rotate, the drive stylet 310. The lead-stylet locking tool 502 may have a proximal tool portion that interfaces with such a knob. In an embodiment, the lead-stylet locking tool 502 include a knob socket (FIG. 17) in the tool body 504, e.g., in the proximal tool portion at the proximal tool end 904.

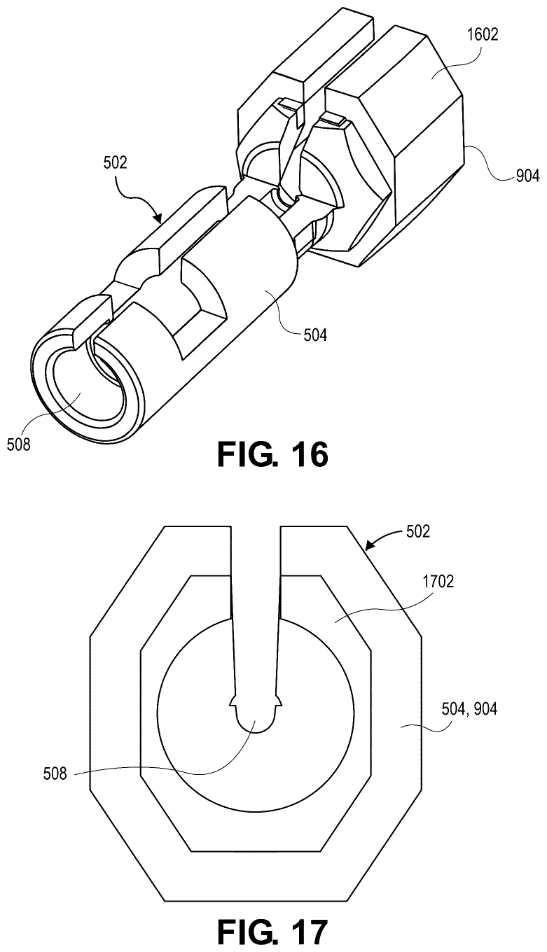

Referring to FIG. 16, a perspective view of a lead-stylet locking tool 502 is shown in accordance with an embodiment. The knob socket at the proximal tool end 904 can extend longitudinally into the proximal tool portion 1602 to form a cavity that can receive the knob on the drive stylet 310. The cavity can have a profile that matches an outer surface profile of the knob. For example, the knob and the knob socket may have a hexagonal shape, as shown. In any case, the knob socket may have a non-circular shape.

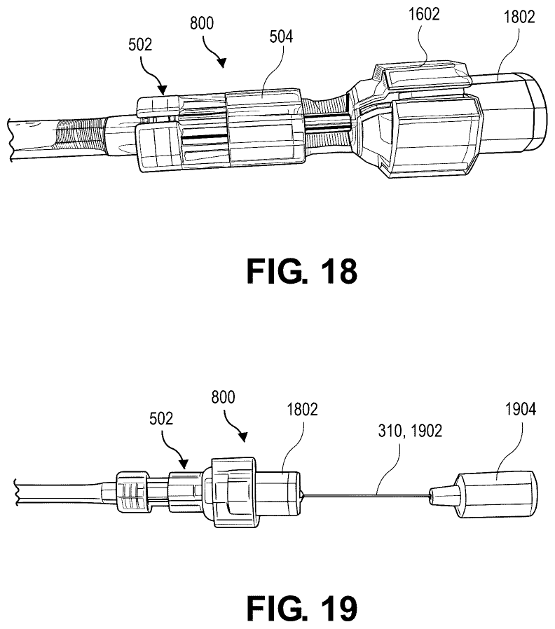

Referring to FIG. 17, an end view of a lead-stylet locking tool 502 is shown in accordance with an embodiment. The drive stylet 310 can include a locking knob (FIG. 18). The locking knob can include an outer surface profile shaped to insert into the knob socket 1702 in the tool body 504. More particularly, the locking knob is shaped to fit within the knob socket 1702 such that the outer profiles of the proximal tool portion 1602 and the locking knob conforms to each other. Such outer profiles can both be non-circular, e.g., hexagonal, to allow the surfaces of the locking knob and the proximal tool portion 1602 to interfere with each other when an attempt at rotating the locking knob is made. More particularly, the knob socket 1702 can receive the locking knob such that the proximal tool portion 1602 resists rotation of the locking knob.

Referring to FIG. 18, a pictorial view of a torque transfer tool 800 is shown in accordance with an embodiment. The proximal tool portion 1602 may also resist longitudinal motion of a locking knob 1802. In an embodiment, the knob socket 1702 is defined by inner wall surfaces of the proximal tool portion 1602 that taper radially inward in the longitudinal direction. When the locking knob 1802 is inserted into the knob socket 1702, the outer surface of the locking knob 1802 can form a press fit against the inner wall surfaces, and the resulting friction fit can resist longitudinal movement, e.g., retraction, of the knob. Accordingly, the locking knob 1802 can be inserted into the knob socket 1702 to achieve one or more of rotational and longitudinal securement of the drive stylet 310 relative to the locking tool 502. The secured bodies, the drive stylet 310, the lead-stylet locking tool 502, and the locking knob 1802, can form the torque transfer tool 800.

Use of a locking knob 1802 design can reduce additional manufacturing requirements to achieve the torque transfer tool 800. More particularly, the locking knob 1802 may be formed in a similar manner to previously used manufacturing processes, including the non-circular outer profile, and therefore the system can use an available drive stylet 310. The locking tool 502 can be shaped to accommodate such drive stylet 310 and achieve the securement between the components.

Referring to FIG. 19, a pictorial view of a torque transfer tool is shown in accordance with an embodiment. When the locking knob 1802 is engaged with the lead-stylet locking tool 502, it may still be necessary or preferable to deliver electrical signals through the stylet body 702 for unipolar pacing. Accordingly, an accessible stylet region may be provided proximal to the locking knob 1802. More particularly, the drive stylet 310 can include a stylet extension 1902 proximal to the locking knob 1802. The stylet extension 1902 can extend proximally from the locking knob 1802, also referred to as first knob herein, to a second knob 1904 of the drive stylet 310. The locking knob 1802 and the second knob 1904 can be longitudinally separated. In an embodiment, the locking knob 1802 and the second knob 1904 are mounted along the stylet body 702. That is, the stylet extension 1902 may be a proximal segment of the stylet body 702. Accordingly, the stylet body 702 may extend continuously from the second knob 1904, through the locking knob 1802, and distally to the stylet tip 312. A physician may therefore utilize the second knob 1904 to manipulate the drive stylet 310, e.g., to insert the locking knob 1802 into the locking socket 1702, and can attach electrical connectors, e.g., a PSA clip, to the stylet extension 1902 during pacing.

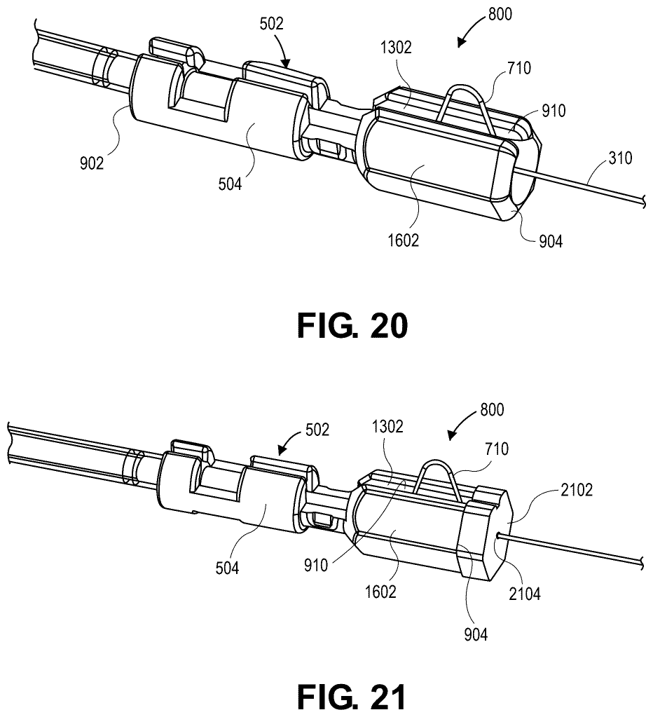

Referring to FIG. 20, a perspective view of a torque transfer tool 800 is shown in accordance with an embodiment. The torque transfer tool 800 can include the lateral space 910 to receive and retain the projection 710 of the drive stylet 310. In an embodiment, the lateral space 910 can be a portion of the side slot 1302, which extend laterally through the tool body 504 to a surrounding environment along an entire length between the proximal tool end 904 and the distal tool end 902. For example, the lateral space 910 can include a segment of the side slot 1302 that extends through the proximal tool portion 1602. In an embodiment, the lateral space 910 has a circumferential width, e.g., between opposing sides of the proximal tool portion 1602 facing each other across the lateral space 910, that is the same or slightly larger than a diameter of the drive stylet 310. Accordingly, when the drive stylet 310 is inserted into the lead-stylet locking tool 502 to form the torque transfer tool 800, the protrusion can slide into the lateral space 910. The close fit of the protrusion within the lateral space 910 allows the opposing sides to resist rotation of the protrusion and the drive stylet 310 in the engaged state.

Optionally, the width of the lateral space 910 is slightly smaller than the stylet diameter to cause a friction fit between the proximal tool portion 1602 and the protrusion in the engaged state. Accordingly, the lead-stylet locking tool 502 can resist both rotational and longitudinal movement of the drive stylet 310, securing the components to form the torque transfer tool 800 in a two-piece system.

Referring to FIG. 21, a perspective view of a torque transfer tool 800 is shown in accordance with an embodiment. An alternative to forming a friction fit between the lead-stylet locking tool 502 and the drive stylet 310 is an end cap 2102 to resist longitudinal motion of the projection 710. More particularly, the lead-stylet locking tool 502 can include an end cap 2102 mounted on the proximal tool end 904 of the proximal tool portion 1602, and the end cap 2102 can hold the projection 710 in place within the lateral space 910. The end cap 2102 can include a central hole 2104 aligned with the tool lumen 508 of the locking tool 502. The end cap 2102 resists retraction of the drive stylet 310 because the projection 710 will contact and be impeded by a distal end of the end cap 2102, which is secured to the proximal tool end 904. Accordingly, the end cap 2102 provides an anti-translation feature to cause the drive stylet 310 to remain fixed in place.

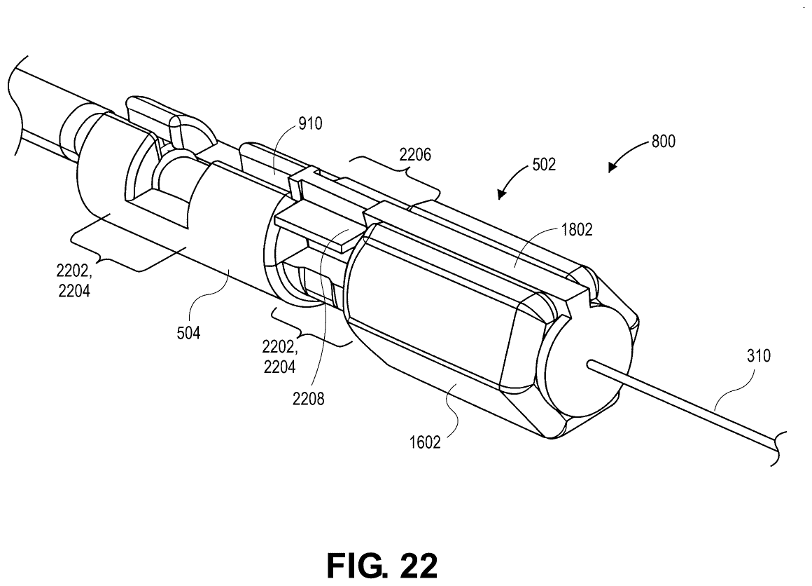

Referring to FIG. 22, a perspective view of a torque transfer tool 800 is shown in accordance with an embodiment. The lateral space 910 can include an intermittent slot 2202 having several slot segments 2204. Such intermittent slots 2202 may be longitudinally separated from each other by a connector opening 2206. The intermittent slots 2202 and connector opening 2206 are described in U.S. Patent Publication No. 2023/0347142, which is referenced above and the contents of which are incorporated by reference herein. During a pacing procedure, the connector opening 2206 can receive an electrical clip, e.g., a PSA clip, to perform pacing or sensing through the pin connector that is exposed through the connector opening 2206.

During lead delivery, the connector opening 2206 can secure the lead-stylet locking tool 502 relative to the drive stylet 310. For example, the drive stylet 310 can include the locking knob 1802 mounted on the stylet body 702, and the locking knob 1802 can engage the tool body 504 surfaces defining the connector opening 2206. In an embodiment, the locking knob 1802 of the drive stylet 310 includes a deployable flap 2208. The deployable flap 2208 can actuate, e.g., flex upward and downward, to reduce an overall width of the locking knob 1802. More particularly, when the deployable flap 2208 is in a first state, it is flexed upward and can slide through the narrow channel of the lateral space 910 in the proximal tool portion 1602. When the deployable flap 2208 is located within the connector opening 2206, it can flex downward, to a second state, to fill a portion of the connector opening 2206, as shown in FIG. 22. In the downward position, the deployable flap 2208 can also interfere with the proximal tool portion 1602. The expanded deployable flap 2208 can prevent the drive stylet 310 from translating relative to the lead-stylet locking tool 502. The proximal tool portion 1602 can resist backout of the deployable flap 2208 and, thus, the deployable flap 2208 can secure the locking knob 1802 in place. Furthermore, the locking knob 1802 can be fixed to the stylet body 702, e.g., by overmolding the locking knob 1802 onto the stylet body 702. Accordingly, the locking knob 1802 can secure the drive stylet 310 within the lead-stylet locking tool 502 to form the torque transfer tool 800 when the deployable flap 2208 is disposed within the connector opening 2206.

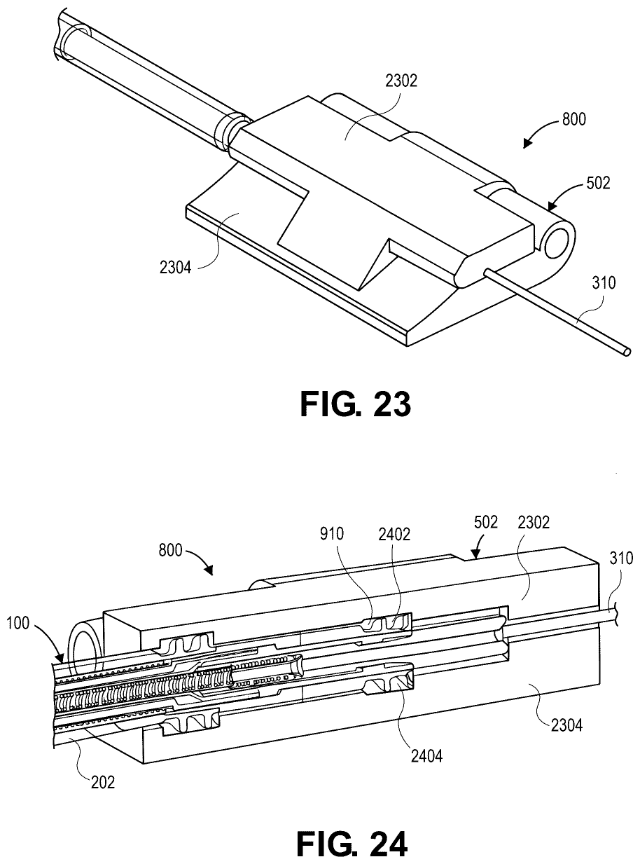

Referring to FIG. 23, a perspective view of a torque transfer tool 800 loaded on an implantable lead 100 is shown in accordance with an embodiment. The two-piece torque transfer tool 800 can include a clamshell-type locking tool 502 that mounts on the drive stylet 310 to hold the drive stylet 310 in place. The clamshell locking tool 502 can have two portions that come together to compress the connector pin 306 and the drive stylet 310.

In an embodiment, the clamshell locking tool 502 includes a first tool portion 2302 and a second tool portion 2304. Similar to the split cap design of FIGS. 11-12, the portions can have respective cavities that combine to form the lateral space 910. The first tool portion 2302 can be hinged to the second tool portion 2304 to allow the clamshell locking tool 502 to move between an open state (not shown) and a closed state (FIG. 23). The clamshell locking tool 502 can be loaded onto the drive stylet 310 in the open state and then actuated to the closed state to grip the drive stylet 310.

Referring to FIG. 24, a sectional view of a torque transfer tool 800 loaded on an implantable lead 100 is shown in accordance with an embodiment. When the clamshell locking tool 502 is actuated to the closed state, a snap fit between the first portion and the second portion can be formed. The snap fit can allow a user to tactically feel the accessory click into place on the drive stylet 310.

In an embodiment, the first tool portion 2302 has a first cavity 2402 and the second tool portion 2304 has a second cavity 2404. In the closed configuration, the first tool portion 2302 combines with the second tool portion 2304 to cause the first cavity 2402 and the second cavity 2404 to form the lateral space 910. The lateral space 910 can secure the drive stylet 310. Also, the combined volume of the cavities 2402, 2404 can provide the channel segments that receive the implantable lead 100, including the pin connector and the lead body 202. Accordingly, the clamshell locking tool 502 can squeeze the implantable lead 100 and the drive stylet 310 to lock the entire system relative to each other and allow the physician to directly transmit torque to the helical fixation element 108 to burrow the lead to the target tissue.

Optionally, the clamshell locking tool 502 can include a latch to lock the first tool portion 2302 to the second tool portion 2304 in the closed state. Accordingly, the clamshell locking tool 502 can grip and hold the drive stylet 310 to form the torque transfer tool 800 and, advantageously, may do so without requiring a specialized wire form. More particularly, the clamshell locking tool 502 may grip a drive stylet 310 that includes or does not include the projection 710.

An aspect of the embodiments relates to a drive stylet 310 comprising a stylet body 702 extending along a longitudinal axis 704 to a stylet tip 312, and a projection 710 extending laterally outward from the stylet body 702.

In an embodiment, the projection 710 is longitudinally compressible.

In an embodiment, the projection 710 includes a distal projection portion 712 joined to a proximal projection portion 714 radially outward from the longitudinal axis 704.

In an embodiment, the distal projection portion 702 and the proximal projection portion 704 join at a projection apex 716 to form a V-shape.

In an embodiment, the stylet tip 312 includes a driver head 706.

Another aspect of the embodiments relates to a lead-stylet locking tool 502. The lead-stylet locking tool 502 comprises a tool body 504 including a body wall 506 extending around a central lumen 508. The central lumen 508 extends along a longitudinal axis 704 from a distal tool end 902 to a proximal tool end 904. The central lumen 508 includes a connector body subsegment 903 sized to receive a connector body of an implantable lead 100 and a pin subsegment 906 sized to receive a connector pin 306 of the implantable lead 100. A lateral space 910 extends through the body wall 506 from the central lumen 508 to receive a projection 710 of a drive stylet 310.

In an embodiment, the lateral space 910 extends through the body wall 506 from the central lumen 508 to receive a projection 710 of the drive stylet 310 as defined according to above.

In an embodiment, the lateral space 910 includes a side slot 1302 extending through the body wall 506 from the central lumen 506 to a surrounding environment.

In an embodiment, the lateral space 910 includes a retaining slot 1304 to receive the projection 710. The retaining slot 1304 has a circumferential slot segment and a longitudinal slot segment.

In an embodiment, the lead-stylet locking tool 502 further comprises an end cap 2102 mounted on the proximal tool end 904. The end cap 2102 includes a central hole 2104 aligned with the central lumen 508.

In an embodiment, the connector body subsegment 903 is sized to receive the connector body 202 in a first friction fit, and the pin subsegment 906 is sized to receive the connector pin 306 in a second friction fit that is tighter than the first friction fit.

A further aspect of the embodiments relates to a torque transfer tool 800. The torque transfer tool 800 comprises a drive stylet 310 including a stylet body 702 extending along a longitudinal axis 704 to a stylet tip 312, and a projection 710 extending laterally outward from the stylet body 702. The torque transfer tool 800 also comprises a lead-stylet locking tool 502 including a tool body 504 having a body wall 506 extending around a central lumen 508. The central lumen 508 extends along the longitudinal axis 704 from a distal tool end 902 to a proximal tool end 904. The central lumen 508 includes a connector body subsegment 903 sized to receive a connector body of an implantable lead 100, and a pin subsegment 906 sized to receive a connector pin 306 of the implantable lead 100. A lateral space 910 extends through the body wall 506 from the central lumen 508 to receive the projection 710 of the drive stylet 310.

In an embodiment, the torque transfer tool 800 comprises the drive stylet 310 as defined according to above and the lead-stylet locking tool 502 as defined according to above.

In an embodiment, the projection 710 includes a distal projection portion 712 joined to a proximal projection portion 714 radially outward from the longitudinal axis 704.

In an embodiment, the tool body 504 is in conforming contact with the projection 710.

In an embodiment, the torque transfer tool 800 further comprises an encapsulant 1004 surrounding the projection 710 within the tool body 504.

In an embodiment, the connector body subsegment 903 is sized to receive the connector body in a first friction fit, and the pin subsegment 906 is sized to receive the connector pin 306 in a second friction fit that is tighter than the first friction fit.

In an embodiment, the lateral space 910 includes an intermittent slot 2202 having a plurality of slot segments 2204 longitudinally separated by a connector opening 2206. The drive stylet 310 includes a deployable flap 2208 disposed within the connector opening 2206.

In an embodiment, the lead-stylet locking tool 502 includes a knob socket 1702 in the tool body 504 at the proximal tool end 904. The drive stylet 310 includes a first knob 1802 shaped to fit within the knob socket 1702.

In an embodiment, the drive stylet 310 includes a second knob 1904. The first knob 1702 and the second knob 1904 are longitudinally separated along the stylet body 702.

In an embodiment, the lead-stylet locking tool 502 includes a first tool portion having a first cavity and a second tool portion having a second cavity. The first tool portion combines with the second tool portion to cause the first cavity and the second cavity to form the lateral space 910.

In an embodiment, the first tool portion is hinged to the second tool portion.

In the foregoing specification, the invention has been described with reference to specific exemplary embodiments thereof. It will be evident that various modifications may be made thereto without departing from the broader spirit and scope of the invention as set forth in the following claims. The specification and drawings are, accordingly, to be regarded in an illustrative sense rather than a restrictive sense.

What is claimed is:

1. A drive stylet, comprising:
a stylet body extending along a longitudinal axis to a stylet tip; and
a projection extending laterally outward from the stylet body.

2. The drive stylet of claim 1, wherein the projection is longitudinally compressible.

3. The drive stylet of claim 2, wherein the projection includes a distal projection portion joined to a proximal projection portion radially outward from the longitudinal axis.

4. The drive stylet of claim 3, wherein the distal projection portion and the proximal projection portion join at a projection apex to form a V-shape.

5. The drive stylet of claim 1, wherein the stylet tip includes a driver head.

6. A lead-stylet locking tool, comprising:
a tool body including a body wall extending around a central lumen, wherein the central lumen extends along a longitudinal axis from a distal tool end to a proximal tool end, wherein the central lumen includes a connector body subsegment sized to receive a connector body of an implantable lead and a pin subsegment sized to receive a connector pin of the implantable lead, and wherein a lateral space extends through the body wall from the central lumen to receive a projection of a drive stylet.

7. The lead-stylet locking tool of claim 6, wherein the lateral space includes a side slot extending through the body wall from the central lumen to a surrounding environment.

8. The lead-stylet locking tool of claim 6, wherein the lateral space includes a retaining slot to receive the projection, and wherein the retaining slot has a circumferential slot segment and a longitudinal slot segment.

9. The lead-stylet locking tool of claim 6 further comprising an end cap mounted on the proximal tool end, wherein the end cap includes a central hole aligned with the central lumen.

10. The lead-stylet locking tool of claim 6, wherein the connector body subsegment is sized to receive the connector body in a first friction fit, and wherein the pin subsegment is sized to receive the connector pin in a second friction fit that is tighter than the first friction fit.

11. A torque transfer tool, comprising:
a drive stylet including a stylet body extending along a longitudinal axis to a stylet tip, and a projection extending laterally outward from the stylet body; and
a lead-stylet locking tool including a tool body having a body wall extending around a central lumen, wherein the central lumen extends along the longitudinal axis from a distal tool end to a proximal tool end, wherein the central lumen includes a connector body subsegment sized to receive a connector body of an implantable lead, and a pin subsegment sized to receive a connector pin of the implantable lead, and wherein a lateral space extends through the body wall from the central lumen to receive the projection of the drive stylet.

12. The drive stylet of claim 11, wherein the projection includes a distal projection portion joined to a proximal projection portion radially outward from the longitudinal axis.

13. The torque transfer tool of claim 11, wherein the tool body is in conforming contact with the projection.

14. The torque transfer tool of claim 11 further comprising an encapsulant surrounding the projection within the tool body.

15. The torque transfer tool of claim 11, wherein the connector body subsegment is sized to receive the connector body in a first friction fit, and wherein the pin subsegment is sized to receive the connector pin in a second friction fit that is tighter than the first friction fit.

16. The torque transfer tool of claim 11, wherein the lateral space includes an intermittent slot having a plurality of slot segments longitudinally separated by a connector opening, and wherein the drive stylet includes a deployable flap disposed within the connector opening.

17. The torque transfer tool of claim 11, wherein the lead-stylet locking tool includes a first knob socket in the tool body at the proximal tool end, and wherein the drive stylet includes a knob shaped to fit within the knob socket.

18. The torque transfer tool of claim 17, wherein the drive stylet includes a second knob, and wherein the first knob and the second knob are longitudinally separated along the stylet body.

19. The torque transfer tool of claim 11, wherein the lead-stylet locking tool includes a first tool portion having a first cavity and a second tool portion having a second cavity, and wherein the first tool portion combines with the second tool portion to cause the first cavity and the second cavity to form the lateral space.

20. The torque transfer tool of claim 19, wherein the first tool portion is hinged to the second tool portion.
